(19) United States
(12) Patent Application Publication
(10) Pub. No.: US 2026/0166488 A1
(43) Pub. Date: Jun. 18, 2026

(54) OPTIMIZING CONDITIONS FOR ANTIBODY PURIFICATION

(71) Applicant: Bayer Healthcare LLC, Whippany, NJ (US)

(72) Inventors: Hendri TJANDRA, Union City, CA (US); Florence RUSLY, Richmond, CA (US)

(21) Appl. No.: 19/126,400

(22) PCT Filed: Nov. 3, 2022
(86) PCT No.: PCT/US2022/048805

§ 371 Date: May 1, 2025

(51) Int. Cl. B01D 61/20; B01D 61/02; B01D 61/14; B01D 61/18; B01D 63/04; B01D 69/08; C07K 1/22; C07K 16/00

(52) U.S. Cl. CPC B01D 61/20; B01D 61/027; B01D 61/145; B01D 61/18; B01D 63/04; B01D 69/08; C07K 1/22; C07K 16/00; C07K 2317/73

(57) ABSTRACT

What is described herein relates to a method for optimizing purification conditions of a protein of interest comprising: a) prediction of filtration performance of a protein of interest based on the hydrophobicity of said protein of interest and/or b) filtration of a protein of interest wherein the protein of interest has an isoelectric point in the range of between 3.0 and 7.5 and the filtration is earned out at a pH above the isoelectric point.

### BACKGROUND FOR THE INVENTION

Biological therapeutics or biologics e.g. recombinant proteins and monoclonal antibodies (mAbs) are usually manufactured by expression in cell cultures i.e. in host cells. Modern purification processes for proteins of interest do not only have to produce a safe and efficacious product reliably and consistently at high yields but require a short bench-to-clinical-trial timeline. There is an increasing pressure for a shorter timeline to develop a scalable manufacturing process for a given protein of interest. The purification process itself must also be as short as possible to achieve an overall economically viable manufacturing process. Hence choosing the optimal purification steps and materials for a given protein of interest must be accomplished as soon as possible and the chosen materials and conditions must allow a fast purification process that yields high product titers.

Therefore, there is a need for a systematic workflow or system to reliably and quickly determine the optimal conditions for purification steps of a given protein of interest. This need can be met by a method for optimizing purification conditions of a protein of interest comprising:
a) prediction of filtration performance of a protein of interest based on the hydrophobicity of said protein of interest and/or
b) filtration of a protein of interest wherein the protein of interest has an isoelectric point in the range of between 3.0 and 7.5 and the filtration is carried out at a pH above the isoelectric point.

### DESCRIPTION OF THE FIGURES

FIG. 1 depicts an exemplary HIC-HPLC chromatogram here of antibody D, which was generated as described in Example 1a below.

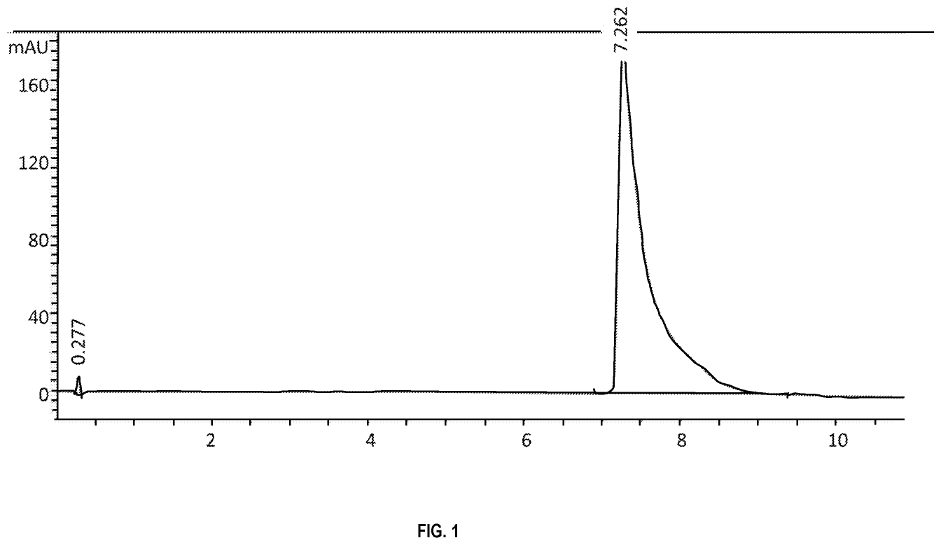

FIG. 2 depicts an exemplary RPLC chromatogram here of antibody C, which was generated as described in Example 1b below.

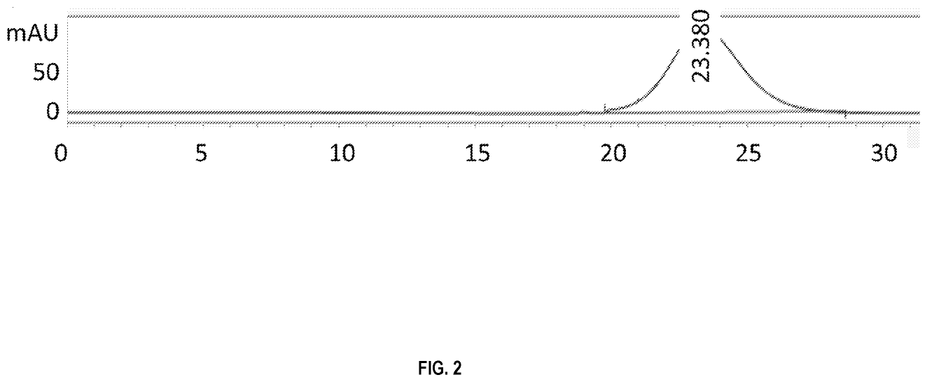

FIG. 3 shows that Antibody C showed an irregular flux pattern in different runs using the same PVDF comprising hollow fiber viral filtration membrane type. Antibody C was expressed by the same CHO cell line in two different bioreactors at 2-10 liter scale. The cell culture fluid harvested via depth filtration from the two bioreactor runs comprising antibody C was further purified prior to filtration using 3 cm² BioEX Polyvinylidene difluoride (PVDF) comprising hollow fiber viral filtration membranes. It can be seen that the first filtration (top grey dashed bar) showed a regular flux pattern i.e. a straight line whereas the subsequent runs all showed a less favorable flux (bars represented by crosses, dashed black line, gray and black line respectively).

FIG. 4 shows the results of FIG. 3 compared to filtering the same cell culture material comprising antibody C employed in FIG. 3 above also through a 10 cm² Planova 20N cellulose comprising hollow fiber viral filter membrane. The bars starting at 30 LMH (thicker gray and thicker black line, respectively) depict the runs using Planova 20N cellulose comprising hollow fiber viral filter membranes whereas the other bars depict the runs using a BioEX Polyvinylidene difluoride (PVDF) comprising hollow fiber viral filtration membranes. This set of observations show that, for a given feedstream, the performance of viral filtration does not necessarily improve simply by using a different viral membrane chemistry. Hence this result demonstrated that the irregular flux pattern was not due to an interaction between antibody C and the hollow fiber membrane chemistry as the flux pattern remained irregular independently of the hollow fiber membrane chemistry i.e. with both cellulose based hollow fibers and PVDF based hollow fiber membranes.

FIG. 5 shows viral filtration flux when the starting material comprising antibody C used in the experiments of FIG. 3 and FIG. 4 above was further purified using a cation exchange membrane. Additional polishing of the materials led to a higher filtration flux. This result confirmed the finding of FIG. 4 that the irregular flux pattern was not due to an interaction between antibody C and the hollow fiber membrane chemistry type; it could be alleviated by removal of impurities from the Feedstream to be filtered.

FIG. 6 shows the results of a filtration of antibody H that had an isoelectric point of 6.5 as determined by iCIEF (imaged capillary isoelectric focusing). A starting feedstream at either pH 5.3 or pH 7.6 was processed through a 10 cm² Planova 20N cellulose comprising hollow fiber viral filter membrane. At pH 5.3-below the isoelectric point—the filter rapidly clogged, thus preventing meaningful filtration. In contrast, filtration at pH 7.6—above the isoelectric point of the mAb-produced a 40-60 LMH flux that could be easily integrated into a scalable manufacturing process.

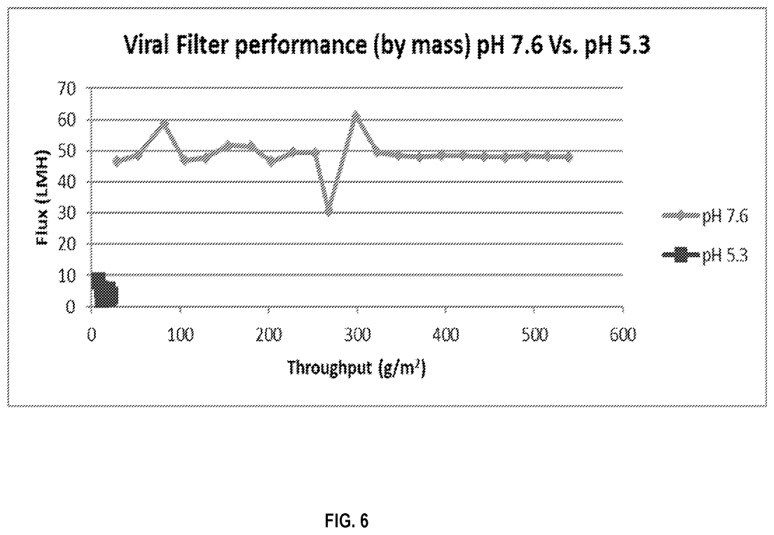

FIG. 7 shows that lowering the conductivity prior to filtering antibody H through a 10 cm² Planova 20N cellulose comprising hollow fiber viral filter membrane at a pH below the isoelectric point-here at pH 4.4-lead to 40-60 LMH flux, but at a considerable loss in productivity compared to filtration through a 10 cm² Planova 20N cellulose comprising hollow fiber viral filter membrane at a pH above the isoelectric point, here filtration at pH 7.0. The productivity is expressed as grams of protein of interest filtered per m² filter per hour of processing. Antibody H had an isoelectric point of 6.5. Hence increasing the pH alone above the isoelectric point had the advantage—as compared to lowering a combination of the pH, protein concentration and conductivity at the lower pH filtration—that the productivity is three times higher. Thus, the filtration process at a pH above the pI was more cost-effective due to the higher productivity. It was also more efficient as the time and material required for dilution prior to viral filtration and subsequent up-concentration following the viral filtration were saved if the viral filtration was carried out at a pH above its isoelectric point.

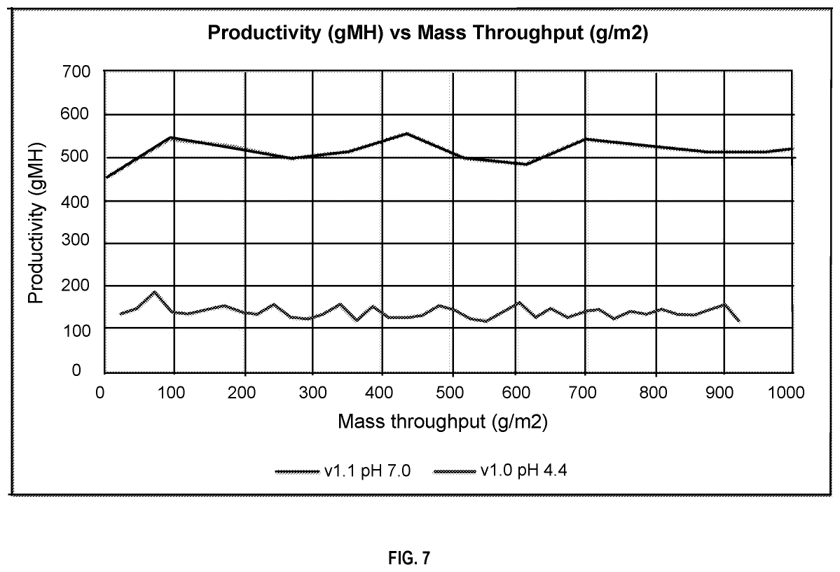

FIG. 8 shows the results of a filtration of antibody H that was determined as hydrophobic (cf. Tables 1 and 2) using a 10 cm² Planova 20N cellulose comprising hollow fiber viral filter membrane (top graph—solid black line) and a 3 cm² BioEX Polyvinylidene difluoride (PVDF) comprising hollow fiber viral filtration membranes (bottom graph, dotted line). It can be clearly seen that knowing the relative hydrophobicity value of the antibody enables us to choose the required viral filtration membrane for purification and thus, in turn, facilitates a more efficient process development. The filtrations were carried out at a pH below the pI of the antibody.

FIG. 9 shows the results of a filtration of antibody F that was determined as hydrophobic (cf. Tables 1 and 2) using a 10 cm² Planova 20N cellulose comprising hollow fiber viral filter membrane (top graph—solid black line) and a 3 cm² BioEX Polyvinylidene difluoride (PVDF) comprising hollow fiber viral filtration membranes (bottom graph, dotted line). It can be clearly seen that knowing the relative hydrophobicity value of the antibody enables us to use the required viral filtration membrane for purification and thus, in turn, facilitates a more efficient process development.

FIG. 10 depicts an exemplary imaged capillary isoelectric focusing (iCIEF) chromatogram of a protein of interest. In this exemplary case the isoelectric point, pI, of the protein of interest would be said to correspond to the main iCIEF peak i.e. would be said to be “E.4”.

FIG. 11 depicts the results of a filtration of antibody I that had an isoelectric point in the range of between 3.0 and 7.5 as determined by iCIEF (imaged capillary isoelectric focusing) as detailed in example 3. A starting feedstream at a pH either below or above the isoelectric point was processed through a 10 cm² Planova 20N cellulose comprising hollow fiber viral filter membrane. Filtration at a pH above the isoelectric point of monoclonal antibody I (cf. grey crosses) resulted in an increased flux compared to filtration at a pH below the isoelectric point of monoclonal antibody I (cf. black triangles). Hence also for a protein of interest with an isoelectric point in the range of between 3.0 and 7.5 which was not determined to be hydrophobic (cf. Tables 1 and 2) a viral filtration at a pH above the isoelectric point was advantageous.

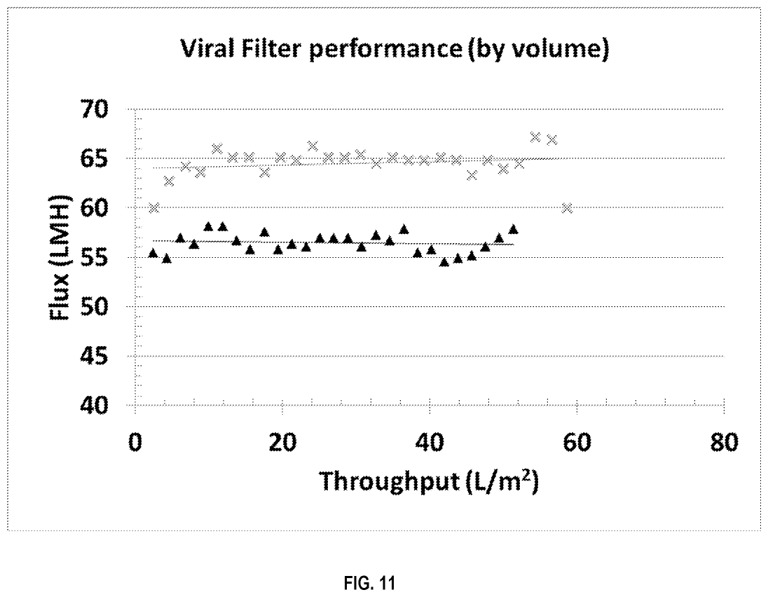

FIG. 12 depicts the results of a filtration of antibody I that had an isoelectric point in the range of between 3.0 and 7.5 as determined by iCIEF (imaged capillary isoelectric focusing) as detailed in example 3. A starting feedstream at a pH either below or above the isoelectric point was processed using a 3 cm² BioEX Polyvinylidene difluoride (PVDF) comprising hollow fiber viral filtration membrane. Filtration at a pH above the isoelectric point of monoclonal antibody I (cf. black squares) resulted in an increased flux compared to filtration at a pH below the isoelectric point of monoclonal antibody I (cf. grey circles). Hence also for a protein of interest with an isoelectric point in the range of between 3.0 and 7.5 which was not determined to be hydrophobic (cf. Tables 1 and 2) a viral filtration at a pH above the isoelectric point was advantageous, irrespective of whether a cellulose comprising hollow fiber viral filtration membrane or a cellulose comprising hollow fiber viral filter membrane was employed.

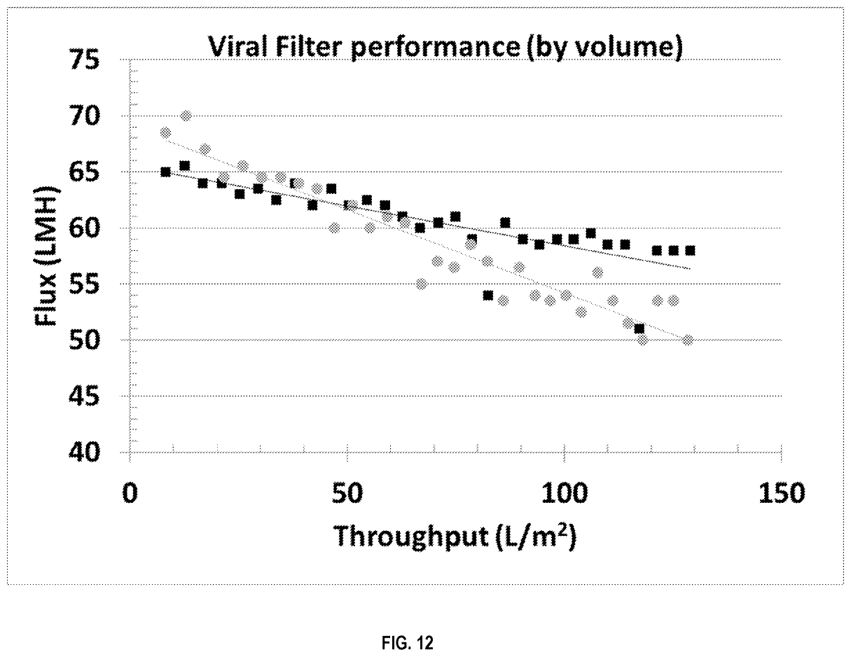

FIG. 13 depicts the results of a filtration of antibody H that had an isoelectric point of 6.5 as determined by iCIEF (imaged capillary isoelectric focusing). A starting feedstream at either pH 4.4 (grey bar) or pH 7.0 (black bar) was processed through a 10 cm² BioEX PVDF comprising hollow fiber viral filter membrane. At pH 4.4—below the isoelectric point—the filter rapidly clogged, thus preventing meaningful filtration. In contrast, filtration at pH 7.0-above the isoelectric point of the mAb-produced a steady filtration flux.

### DETAILED DESCRIPTION

Described herein is a method for optimizing purification conditions of a protein of interest comprising
a) prediction of filtration performance of a protein of interest based on the hydrophobicity of said protein of interest and/or
b) filtration of a protein of interest wherein the protein of interest has an isoelectric point in the range of between 3.0 and 7.5 and the filtration is carried out at a pH above the isoelectric point.

As well as related aspects.

In a preferred embodiment the filtration according to part a) of the method for optimizing purification conditions described herein is a viral filtration.

Part a) of the method for optimizing purification conditions of a protein of interest is based on the finding that it is possible to choose optimal viral filtration membranes via determining the hydrophobicity of the protein of interest.

For economic reasons industrial-scale viral filtration membranes are routinely chosen based on factors such as low differential pressure and high volumetric throughput at which the respective membranes can be operated and whether an overall short processing time is facilitated by the respective membrane. When process optimization is required, the pH and/or the conductivity of a filtration step are usually evaluated. Additionally, the interaction between the filter chemistry and any impurities present together with the protein of interest can be considered when selecting a suitable viral filter membrane. Optimizing the choice of viral filtration membranes can be very time- and material-consuming as viral filtration is typically incorporated in the penultimate stage of a downstream manufacturing process. Thus, even if a bench scale-down model is available, the required volumes and amount of the starting harvest material needed for such studies could be relatively large. It follows that the materials and equipment required for process optimization experiments can be prohibitively expensive and time-consuming to execute. However, it is now possible to choose optimal viral filtration membranes i.e. viral filters based on the hydrophobicity of the protein of interest. Hence it is, in turn, possible to quickly choose the viral filtration membrane suitable for a given protein of interest reliably and efficiently, thereby accelerating process development.

To a person skilled in the art it is clear that the method for optimizing purification conditions and especially for choosing the optimal viral filtration membrane by measuring the hydrophobicity of the protein of interest described herein could also be employed for choosing a tangential flow filtration membrane.

In one embodiment of part a) of the method for optimizing purification conditions of a protein of interest described herein the prediction of viral filtration performance of a protein of interest based on the hydrophobicity of said protein of interest comprises
1) Determining the hydrophobicity of the protein of interest
2) Choosing a cellulose comprising hollow fiber viral filter membrane or a Polyvinylidene difluoride (PVDF) comprising hollow fiber viral filter membrane based on the hydrophobicity of the protein of interest.

Examples for cellulose comprising hollow fiber viral filter membranes are Planova 15N, Planova 20N and Planova 35N. In one embodiment of part a) of the method for optimizing purification conditions of a protein of interest described herein the cellulose comprising hollow fiber viral filter membrane is Planova 20N.

Examples for Polyvinylidene difluoride (PVDF) comprising hollow fiber viral filter membranes are Planova BioEx, Viresolve NFP, Ultipor DV20, Ultipor DV50 and Pegasus SV4 (Ide, 2022). In one embodiment of part a) of the method for optimizing purification conditions of a protein of interest described herein the Polyvinylidene difluoride (PVDF) comprising hollow fiber viral filter membrane is Planova BioEx.

It is clear to a skilled person, that the value determined for the hydrophobicity of a given protein of interest can be an empirical and/or relative value and/or an absolute value. This is the case as different methods for determining the hydrophobicity exist.

In an embodiment of part a) of the method for optimizing purification conditions of a protein of interest described herein the determined hydrophobicity of the protein of interest is compared to a reference prior to choosing the viral filtration membrane type. Such a reference can for example be a known protein e.g. a monoclonal antibody. Such a comparison has the advantage that as the characteristics of the known antibody have already been determined the hydrophobicity value determined for the protein of interest is even more meaningful if it is compared to a known standard. This is the case for both absolute and relative hydrophobicity values. This is especially the case if the known reference antibody is run in parallel with the protein of interest in the assay that is used for determining hydrophobicity.

In a preferred embodiment of part a) of the method for optimizing purification conditions of a protein of interest described herein the hydrophobicity of the protein of interest is determined using HIC-HPLC and/or RPLC.

Hydrophobic interaction chromatography (HIC) separates molecules based on their hydrophobicity. HIC is a useful separation technique for purifying proteins while maintaining biological activity due to the use of conditions and matrices that operate under less denaturing conditions.

In a preferred embodiment of part a) of the method for optimizing purification conditions of a protein of interest described herein the hydrophobicity of the protein of interest is determined using HIC-HPLC. Depending on the outcome of this HIC-HPLC the protein of interest is classified as less hydrophobic or hydrophobic either based on comparison to a reference protein or based on absolute values.

As already mentioned above it is clear to a skilled person, that the value determined via HIC-HPLC for the hydrophobicity of a given protein of interest can be an empirical and/or relative value and/or an absolute value. In other words, if the protein of interest is classified as less hydrophobic or hydrophobic based on comparison to a reference protein then the behavior of the reference protein can be directly compared to the behavior of the protein of interest under comparable HIC-HPLC conditions i.e. whether the HIC-HPLC main peak retention time of the protein of interest compared to the reference protein shows that it is more or less hydrophobic than the reference protein.

In a preferred embodiment of the method for optimizing purification conditions described herein a given protein of interest is classified as hydrophobic if the retention time of the protein of interest is ≥ retention time of antibody G disclosed as Tpp9252 in WO2020/089380 A1 under comparable HIC-HPLC conditions.

Alternatively, the absolute values of the HIC-HPLC main peak retention time of the reference protein and the protein of interest can be compared as is it shown in Table 1. Moreover, empirical values could be used to classify a protein of interest.

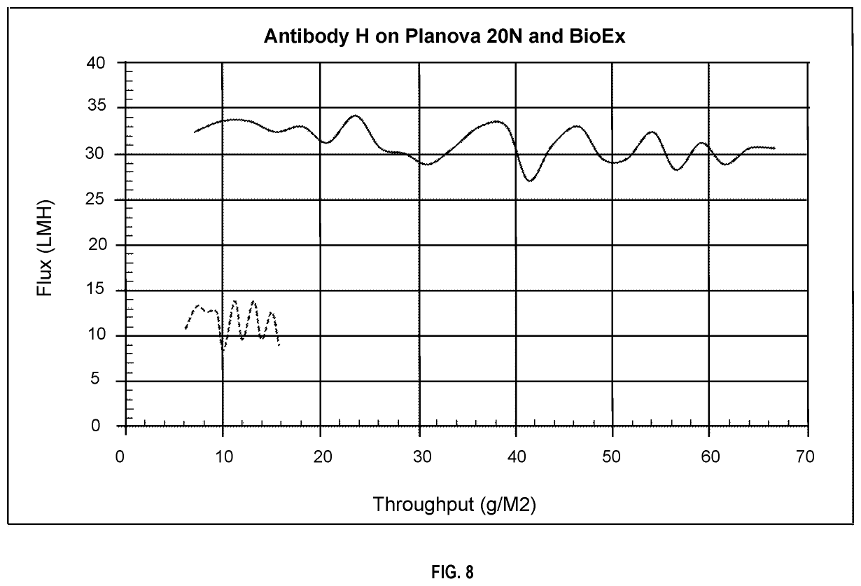

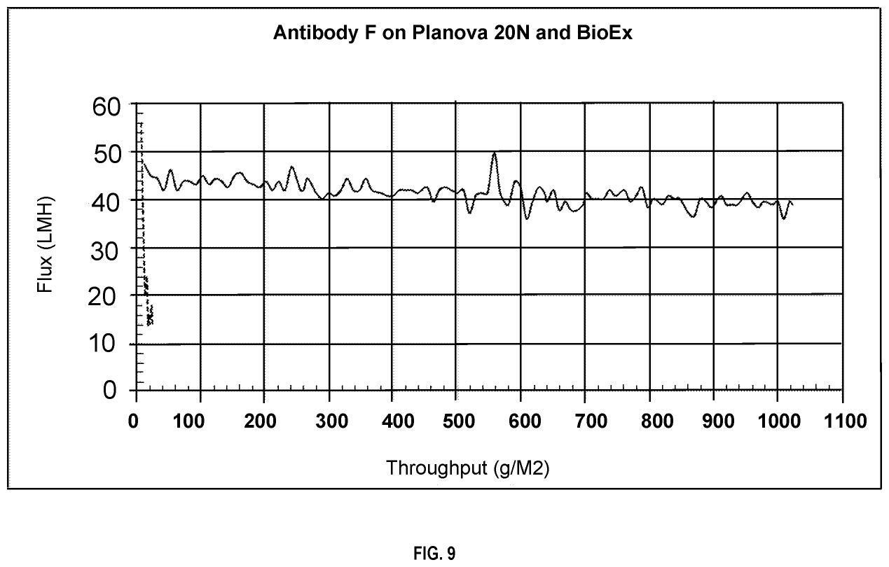

Under the exemplary conditions described example 1A below, a determination based on absolute HIC-HPLC main peak retention time (min) value for any protein of interest can be carried out. Under the given exemplary condition for a protein of interest with an absolute HIC-HPLC main peak retention time (min) value greater than ˜9.7 a cellulose comprising hollow fiber viral filter membranes is advantageous. Whereas if the HIC-HPLC main peak retention time (min) value was lower than ˜9.7 a PVDF comprising hollow fiber viral filtration membrane can be chosen. In other words, if the protein of interest is less hydrophobic a PVDF comprising hollow fiber viral filtration membrane such as the Planova BioEX can be used whereas if the protein of interest is more hydrophobic, then a cellulose comprising hollow fiber viral filtration membrane such as Planova 20N is advantageous (cf. FIGS. 8 and 9).

Apart from cellulose and PVDF comprising hollow fiber viral filtration membranes respectively, other commonly employed viral and tangential flow filtration membranes comprise, Polyethersulfone (PES). Moreover, filtration membranes comprising polytetrafluoroethylene (PTFE), Polypropylene (PP), Cellulose nitrate (CN), Nylone/Polyamide (NYL), cellulose acetate (CA) and Anopore (AN) are known.

In a preferred embodiment of the method for optimizing purification conditions of a protein of interest described herein the filtration according to part b) is a viral filtration.

As for part b) of the method for optimizing viral filtration conditions of a protein of interest described herein the finding that viral filtration outcome of a protein of interest wherein the protein of interest has an isoelectric point in the range of between 3.0 and 7.5 improves if the viral filtration is carried out at pH above the isoelectric point was also unexpected. This was the case since with an isoelectric point in the range of between 3.0 and 7.5 a protein of interest would be positively charged at a pH below its isoelectric point. However, in contrast to what was reported by Hongo-Hirasaki et al., 2010 increasing the ionic strength did not alleviate poor filtration flux performance. On the contrary, increasing ionic strength of the protein feedstream led to multimer formation (data not shown). Instead of adjusting down a combination of ionic strength, protein concentration and pH of a protein solution to improve viral filtration flux (see above), it was observed that adjusting the pH of the protein solution alone to above its isoelectric point led to a significantly improved viral filter performance

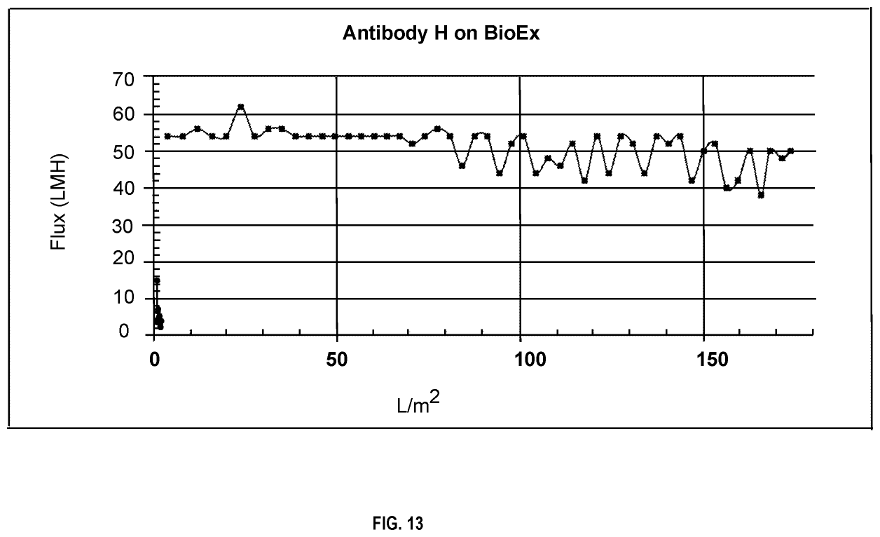

The viral filtration of a protein of interest wherein the protein of interest has an isoelectric point in the range of between 3.0 and 7.5 at pH above the isoelectric point was demonstrated to work with different hollow fiber viral filter membranes e.g. Planova 20N (a cellulose comprising hollow fiber viral filter membrane), S20N and Planova BioEX (a PVDF comprising hollow fiber viral filtration membrane) as well as Merck Millipore Vpro (a Polyethersulfone (PES) comprising hollow fiber viral filtration membrane) were tested (data not shown). Hence viral filtration of a protein of interest wherein the protein of interest has an isoelectric point in the range of between 3.0 and 7.5 at pH above the isoelectric point was shown not to be restricted to the interaction of a specific protein of interest e.g. a monoclonal antibody with a specific membrane type e.g. a cellulose, PVDF or PES comprising membrane (cf. FIG. 13).

In a preferred embodiment of the method for optimizing purification conditions described herein the protein of interest has an isoelectric point in the range of between 3.0 and 7.5 more preferably between 5 and 6.5.

In a preferred embodiment of the method for optimizing purification conditions described herein, the viral filtration is carried out at a pH in the range of between 5.5-8.5.

It is clear to a skilled person, that the isoelectric point (pI) of a given protein of interest can be determined in different ways. For example, several algorithms for estimating isoelectric points of peptides and proteins have been developed, which calculate the isoelectric point based on primary sequence information. Most of them use the Henderson-Hasselbalch equation with different pK values. These predicted pI values can differ from an isoelectric point value determined empirically in a laboratory due to post-translational modifications e.g. glycosylation.

In a preferred embodiment of the method for optimizing purification conditions described herein the isoelectric point of the protein of interest is determined via Imaged Capillary Isoelectric Focusing (icIEF).

The imaged capillary isoelectric focusing (icIEF) separates ampholytic components of biomolecules in an electric field according to their isoelectric points and has been used for protein charge variants quantification and characterization.

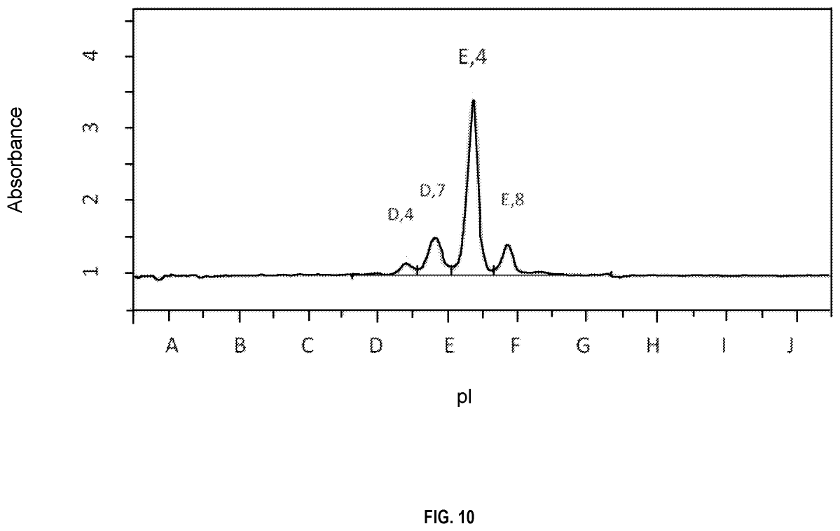

Changes in charge may reflect deamidation, aggregation, isomerization, fragmentation, glycation of lysine residues, and/or glycan sialyation, etc. Conversion from one charged isoform to another indicates a change is occurring to the protein under the conditions being analyzed. An example of an iCIEF chromatogram showing the pH of the various charged variants of an antibody is depicted in FIG. 10.

In a preferred embodiment of the method for optimizing purification conditions described herein the largest peak in the icIEF chromatogram of a given protein of interest is taken to correspond to the pI of that protein of interest.

In a preferred embodiment of the method for optimizing purification conditions described herein in case the protein of interest e.g. a monoclonal antibody is determined to be hydrophobic and to have a low isoelectric point in the range of between 3.0 to 7.5 then viral filtration is carried out using a cellulose comprising hollow fiber viral filter membrane at a pH above the isoelectric point of the protein.

What is described herein also relates to a method for filtration of a protein of interest wherein the protein of interest has an isoelectric point in the range of between 3.0 to 7.5 and filtration is carried out at pH above the isoelectric point.

In a preferred embodiment of this aspect the filtration is a viral filtration.

In another preferred embodiment of this aspect the filtration is a tangential flow filtration.

This is the case as it was found that during tangential flow filtration of protein of interest e.g. a monoclonal antibody with a isoelectric point above 3 but below 7.5 a higher permeate flow rate/higher permeate flux (data not shown) and hence a more efficient process due to minimized processing time could be reached if the monoclonal antibody was filtered at a pH above isoelectric point. This also increase the productivity.

In a preferred embodiment of the method for optimizing purification conditions described herein in case the protein of interest has an isoelectric point in the range of between 3.0 to 7.5 then both the viral filtration and the tangential flow filtration are carried out at a pH above isoelectric point, preferably at a pH in the range of between 5.5-8.5.

In another preferred embodiment of the method for optimizing purification conditions described herein in case the protein of interest has an isoelectric point in the range of between 3.0 to 7.5 then the tangential flow filtration is carried out at a pH above the isoelectric point of the protein of interest, preferably at a pH in the range of between 5.5-8.5, using a cellulose comprising hollow fiber filtration membrane.

In an especially preferred embodiment of the method for optimizing purification conditions described herein in case the protein of interest has an isoelectric point in the range of between 3.0 to 7.5 then both the viral filtration and the tangential flow filtration are carried out at a pH above the isoelectric point of the protein of interest, preferably at a pH in the range of between 5.5-8.5,

In another embodiment of the method for optimizing purification conditions described herein in case the protein of interest has an isoelectric point in the range of between 3.0 to 7.5 and is determined to be hydrophobic then preferably both the viral filtration and the tangential flow filtration are carried out at a pH above the isoelectric point of the protein of interest, preferably at a pH in the range of between 5.5-8.5 using a cellulose comprising hollow fiber filtration membrane

The term flux refers to how fast a fluid (e.g. a cell culture fluid sample) is pumped wherein the value is normalized to each m² of the respective filter. Its unit is liter of cell culture fluid sample per hour per m² (LMH). It is regarded as input parameter for a filtration.

The term throughput describes the volume of a filtrate and, hence, a filtration output. For example, for a particular filtration flow e.g. 70 L/hr for 1 m² filter, the throughout could be 40 L of a cell culture fluid sample per 1 m² of filter at a delta pressure of 0.68 bar (10 psi) and 60 L of the cell culture fluid sample per 1 m² of filter at a delta pressure of 1.03 bar (15 psi). Hence in this case, the throughput of the filter is 40 L/m² and 60 L/m² at dP=0.68 and dP=1.03 bar, respectively. In other words, a higher throughput of a filter is possible at the expense of the higher differential pressure.

Hollow fiber membranes are a class of artificial membranes comprising a semi-permeable barrier in the form of a hollow fiber. Most commercial hollow fiber membranes are packed into cartridges which can be used for a variety of liquid and gaseous separations. The membranes from which the hollow fibers are formed can be of a variety of materials.

In another aspect what is described herein relates to the using the hydrophobicity of a protein of interest to choose a viral filter and/or tangential flow filtration membrane.

In a preferred embodiment of the method for optimizing purification conditions described herein and the use described herein the protein of interest is expressed in a mammalian cell culture.

In a preferred embodiment of the method for optimizing purification conditions described herein and the use described herein the protein of interest is an antibody e.g. a monoclonal antibody.

### REFERENCES

Hongo-Hirasaki, Tomoko et al. (2010) Effect of antibody solution conditions on filter performance for virus removal filter Planova 20N, Biotechnol Prog 2010 July-August; 26 (4): 1080-7.
Ide (2022). Filter made of cuprammonium regenerated cellulose for virus removal: a mini-review Cellulose volume 29, pages 2779-2793

### EXAMPLES

### Example 1. Determining Hydrophobicity of a Given Protein of Interest

In this example the hydrophobicity of different monoclonal antibodies was assessed using both Hydrophobic Interaction Chromatography-High Performance Liquid Chromatography (HIC-HPLC) and Reverse Phase liquid chromatography (RPLC) analysis.

a. HIC-HPLC

The hydrophobicity of different proteins e.g. monoclonal antibodies was assessed via HIC-HPLC analysis. This analysis measured how the hydrophobic components/patches/domains of the proteins were bound and then eluted from the HIC-HPLC column. 50 μg of each sample was analyzed on an Agilent 1200 Series HPLC instrument and TSKgel Butyl-NPR 4.6 mm ID×3.5 cm, 2.5 mm column (Tosoh Bioscience LLC, King of Prussia, PA). Two mobile phase buffers were used in complementary 0-100% gradients: Buffer A: 25 mM Tris-HCL, 1.5 M ammonium sulfate, pH 7.0; Buffer B: 25 mM Tris-HCL, 5% 2-propanol, pH 7.0. Sample flow was 1 mL/mL and a 20-minute total run time. UV detection was performed at Abs280 nm with a bandwidth of 16 and reference correction at 360 nm with a 100 bandwidth.

The results are presented in Table 1 below. The samples were ranked from most hydrophobic to least hydrophobic within a group of evaluated proteins of interest here monoclonal antibodies (mAbs). As the HIC-HPLC Column was eluted in an increasing amount of eluant, the longer retention time reflected the hydrophobicity strength of the proteins bound to the HIC column. The samples are ranked from most hydrophobic to least hydrophobic.

TABLE 1

| Antibody sample | Main Peak Retention Time (min) |
|---|---|
| Antibody F | 12.561 |
| Antibody H | 9.801 |
| Antibody G | 9.744 |
| Protein B | 9.340 |
| Antibody C | 9.094 |
| Antibody I | 8.423 |
| Antibody A | 8.038 |
| Antibody D | 7.262 |

Based on the prior knowledge for antibodies F and H and their relative hydrophobicities to those of other mAbs listed on Table 1, for any antibody with the main peak retention time (min) of greater than ˜9.7, a cellulose comprising hollow fiber viral filter membrane was chosen.

### b) RPLC

In the absence of HIC-HPLC method, the relative hydrophobicity of different proteins e.g. monoclonal antibodies may also be assessed via RPLC Analysis. Approximately 100 μg of each sample was analyzed on an Agilent 1200 Series High Pressure Liquid Chromatography instrument and Poroshell 300SB-C8 column 2.1 mm×75 mm (Agilent) at 75° C. Two mobile phase buffers were used in complementary 0-100% gradients: solvent A: 0.2% TFA in H₂0, solvent B: 0.2% TFA in ΔCN. Sample flow rate was 0.5 mL/min and a 45 min total run time. UV detection was performed at Abs280 nm with a bandwidth of 8 nm.

The RP-HPLC results are presented in Table 2 below. The samples are ranked from most hydrophobic to least hydrophobic.

| Antibody sample | Main Peak Retention Time (min) |
|---|---|
| Antibody H | 25.2 |
| Antibody G | 25.1 |
| Antibody F | 24.9 |
| Protein E | 23.8 |
| Antibody C | 23.4 |
| Antibody D | 23.3 |
| Protein B | 22.9 |
| Antibody A | 22.9 |

### Example 2. Accelerating Process Development Using the Method Described Herein

Antibody C was loaded at constant pressure to generate a starting flux of 60 liter/m²/hr onto a 3 cm² Planova BioEx hollow fiber membrane. An irregular flux was observed in different runs of this setting.

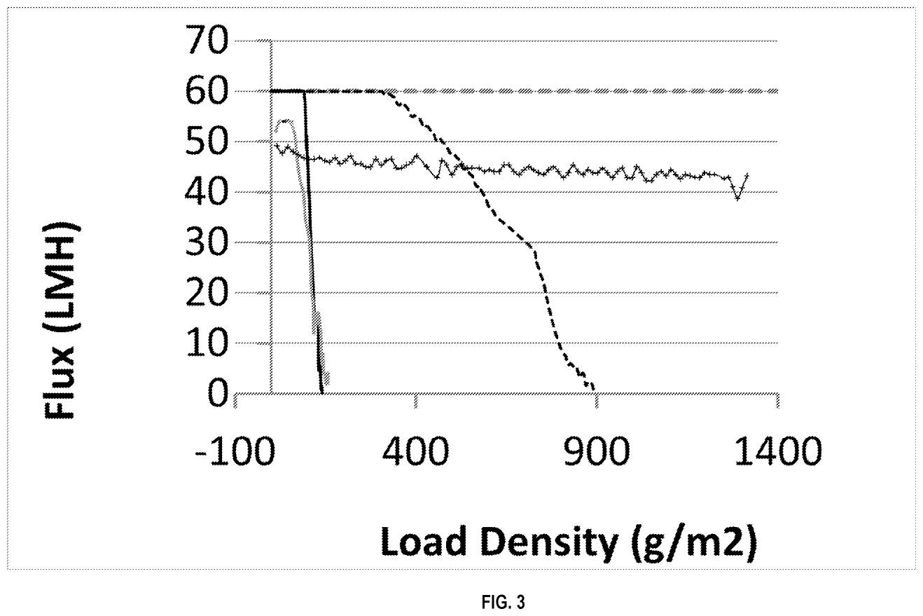

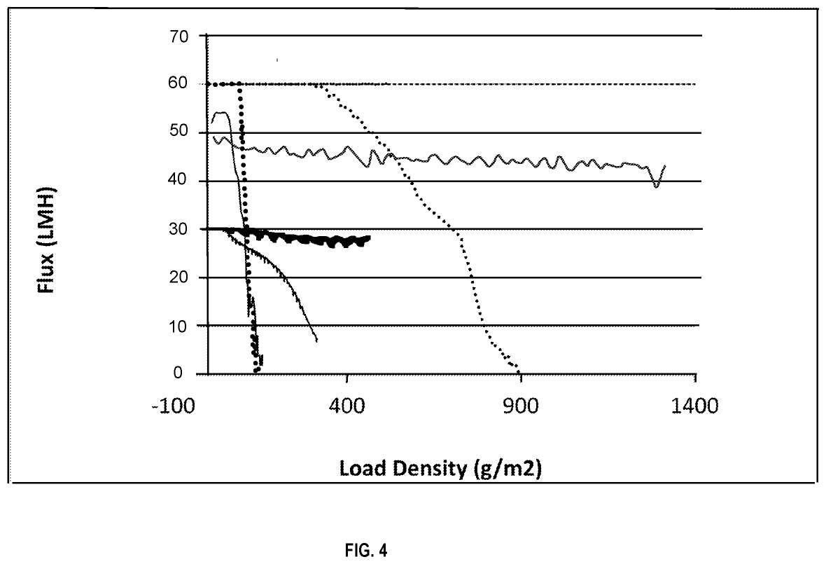

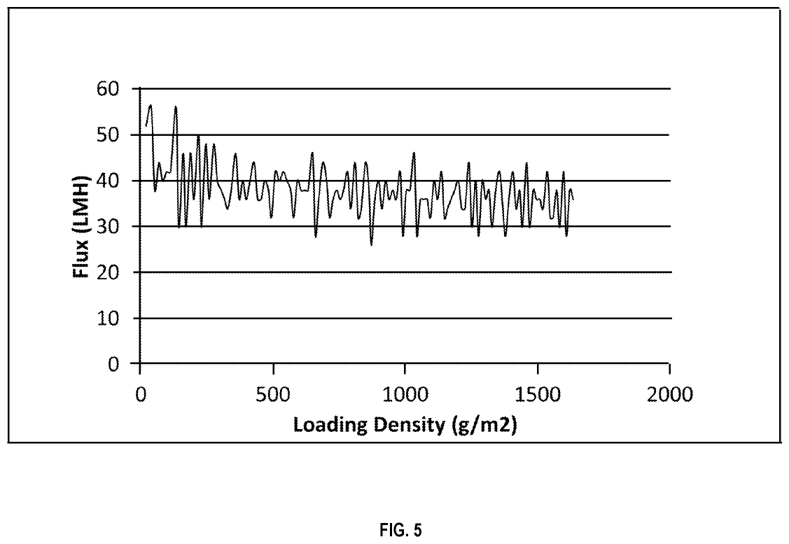

To alleviate the observed flux decay (cf. FIG. 3) several approaches were evaluated. It was inter alia investigated whether the flux decay was due to membrane chemistry of the hollow fiber viral filter (i.e. cellulose vs PDVF) or membrane format (i.e. flat sheet vs hollow fiber) was used. Therefore, the experiment was repeated using different hollow fiber viral filtration membranes: a Planova BioEx hollow fiber viral filtration membrane—i.e. a PVDF comprising hollow fiber viral filtration membrane—a Planova 20N hollow fiber viral filtration membrane—i.e. a membrane comprising regenerated cellulose—a Virosart CPV—i.e. a flat sheet viral filtration PES membrane- and a Pegasus Prime—i.e. another PES flat sheet viral filtration membrane. However, this did not solve the observed flux inconsistency (cf. FIG. 4). It was subsequently found that neither altering pH nor conductivity (data not shown) but an ion exchange chromatography step prior to viral filtration alleviated the problem reliably (cf. FIG. 5). Thus, in retrospect, if the method described herein would have already been known at the time, it would have been concluded from the hydrophobicity of antibody C as given in Tables 1 and 2 above that the observed flux decay could not have been due to employing a PVDF hollow fiber viral filtration membrane. Hence, the time consuming and expensive experiments regarding the different membrane types and formats could have been omitted leading to an overall faster process development.

### Example 3. Improved Filtration Performance at a pH Above Isoelectric Point of Protein of Interest

Antibody H was clarified, affinity-captured and polished before processed on viral filters. The antibody was processed through a 10 cm² Planova 20N cellulose comprising hollow fiber viral filter membrane at a constant pressure of 1 bar at either pH 5.3 or pH 7.6. Experimental controls using 5 cm² Virosart or 3.4 cm² Viresolve Pro filter were processed per vendor recommendations. The studies were carried out at ambient temperature (17-23° C.). Filtrate weight were collected on a balance and filtration time recorded. Flux and throughput were subsequently calculated. At pH 5.3-below the isoelectric point of antibody H—the viral filter rapidly clogged preventing meaningful filtration. In contrast, when same antibody was filtered at pH 7.6—above the isoelectric point of the mAb—a filtration flux of 40-60 LMH was observed.

The isoelectric point of individual antibody H was determined empirically using iCIEF (imaged capillary isoelectric focusing) using the ProteinSimple iCE3 System with Alcott 720NV autosampler unit and iCIEF Cartridge P/N 101701 (Carrier Ampholytes, Pharmalyte 3-10, Cat No. 17-0456-01, GE-Healthcare). The samples were diluted to working range (1-2 mg/mL), mixed with Master Mix (which has 0.35% methyl cellulose, 2.5 M urea, 5% (v/v) of 3 to 10 ampholytes and pI markers (4.22 and 9.46) at 2 μg/mL). The analysis was carried out according to the manufacturers' instructions. The pH of the main peak from such iCIEF analysis corresponds to the empirical isoelectric point.

1. A method for optimizing purification conditions of a protein of interest comprising
a) predicting filtration performance of a protein of interest based on the hydrophobicity of said protein of interest and/or
b) filtering a protein of interest wherein the protein of interest has an isoelectric point in the range of between 3.0 to 7.5 and the filtration is carried out at pH above the isoelectric point.

2. The method according to claim 1 wherein the filtration according to part a) is a viral filtration.

3. The method according to claim 1 wherein the filtering according to part b) is a viral filtration or a tangential flow filtration.

4. The method according to claim 2, wherein the prediction of predicting viral filtration performance of a protein of interest based on the hydrophobicity of said protein of interest comprises
a.1) Determining the hydrophobicity of the protein of interest
a.2) Choosing a cellulose comprising hollow fiber viral filter membrane or a Polyvinylidene difluoride (PVDF) comprising hollow fiber viral filter membrane based on the hydrophobicity of the protein of interest.

5. The method according to claim 2, wherein the determined hydrophobicity of the protein of interest is compared to a reference prior to choosing the viral filter type.

6. The method according to claim 2, wherein the hydrophobicity of the protein of interest is determined using HIC-HPLC and/or RPLC and/or an algorithm for estimating the isoelectric point.

7. The method according to claim 1, wherein the protein of interest is expressed in a mammalian cell culture and is a monoclonal antibody.

8. The method according to claim 2 wherein the filtration is carried out using a hollow fiber module.

9. The method according to claim 8 wherein the hollow fiber module has a pore size of 20 nm.

10. The method according to claim 3 wherein the filtration is carried out at a pH in the range of between 5.5-8.5.

11. The method according to claim 3 wherein the isoelectric point of the protein of interest is determined via Imaged Capillary Isoelectric Focusing (iCIEF)

12. Use of the hydrophobicity of a protein of interest to choose a viral filtration membrane.

13. Filtration of a protein of interest wherein the protein of interest has an isoelectric point in the range of between 3.0 to 7.5 and the filtration is carried out at pH above the isoelectric point.
